(19) United States
(12) Patent Application Publication
(10) Pub. No.: US 2026/0248478 A1
(43) Pub. Date: Aug. 27, 2026

(54) ULTRASOUND SYSTEM FOR ACQUIRING ANGLE-COMPENSATED FLOW INFORMATION USING A THREE-DIMENSIONAL (3D) VASCULAR MODEL GENERATED BASED ON TRACKING DATA OF AN ULTRASOUND PROBE

(71) Applicant: GE Precision Healthcare LLC, Waukesha, WI (US)

(72) Inventors: Jacob OLESEN, Copenhagen (DK); Bo MARTINS, Rodovre (DK); Amalie EKSTRAND, Copenhagen (DK); Anders OLIN, Copenhagen (DK)

(21) Appl. No.: 19/065,420

(22) Filed: Feb. 27, 2025

(51) Int. Cl. A61B 8/06; A61B 8/00; A61B 8/08

(52) U.S. Cl. CPC A61B 8/06; A61B 8/4281; A61B 8/483; A61B 8/5207; A61B 8/54; A61B 8/08

(57) ABSTRACT

An ultrasound system may acquire ultrasound data of a vascular structure of the region of interest of the subject from an ultrasound probe. The ultrasound system may acquire tracking data of the ultrasound probe from a tracking system. The ultrasound system may generate a three-dimensional (3D) vascular model of the vascular structure using the ultrasound data and the tracking data. The ultrasound system may acquire angle-compensated flow information of the vascular structure using a flow imaging technique and the 3D vascular model of the vascular structure. The ultrasound system may display the angle-compensated flow information of the vascular structure.

### TECHNICAL FIELD

The present disclosure relates, generally, to an ultrasound system for acquiring angle-compensated flow information using a 3D vascular model generated based on tracking data of an ultrasound probe. More specifically, the present disclosure relates to an ultrasound system that generates a 3D vascular model of a vascular structure of a region of interest of a subject based on ultrasound data acquired by an ultrasound probe and tracking data of the ultrasound probe, and acquires angle-compensated flow information using the 3D vascular model and a flow imaging technique.

### BACKGROUND

An ultrasound system may generate a B-mode ultrasound image which is a two-dimensional image of a region of interest of a subject and which displays various structures of the region of interest in gray-scale. For instance, the ultrasound system may transmit ultrasound signals towards the region of interest, receive echo signals that are reflected by, or back-scattered from, the region of interest, and generate a B-mode ultrasound image based on the amplitude of the echo signals. In this case, a user may view the B-mode ultrasound image to assess the relevant anatomy of a region of interest of a subject.

In some cases, the ultrasound system may also acquire flow information of a particular region of the region of interest using a flow imaging technique. For example, the ultrasound system may use a power Doppler technique to determine the presence of flow in the particular region of the region of interest, and display a B-mode image that is overlaid with flow information that depicts the presence of flow in the particular region. As another example, the ultrasound system may use a color Doppler technique to determine the velocity and direction of flow in the particular region of the region of interest, and display a B-mode image that is overlaid with flow information that depicts the velocity and direction of flow in the particular region. As yet another example, the ultrasound system may use a vector flow imaging technique to determine the velocity and direction of flow at any point within the particular region of the region of interest, and display a B-mode image that is overlaid with flow information that depicts the velocity and direction of flow at any point within the particular region of the region of interest in the particular region. As yet another example, the ultrasound system may use a spectral Doppler technique to determine the velocity of flow along a particular line (e.g., a continuous-wave (CW) Doppler line or a pulsed-wave (PW) Doppler line) and/or within a particular gate (e.g., a PW Doppler gate) over time.

In general, an ultrasound system may acquire flow information based on a transmit frequency of ultrasound signals, a received frequency of echo signals, and an angle of insonation between an ultrasound beam and the direction of blood flow in a vascular structure. For instance, the ultrasound system may determine a velocity of blood in a vascular structure based on a frequency shift between the transmitted ultrasound signals and the received echo signals and the insonation angle. The determined velocity may, or might not, be accurate based on the particular angle of insonation. For example, an insonation angle of 0° may result in an accurate velocity measurement, whereas insonation angles greater than 60° may result in relatively inaccurate velocity measurements. The ultrasound system may compensate for the insonation angle by performing angle compensation such that the angle-compensated velocity measurement is more accurate.

Ultrasound is a commonly used imaging modality during surgical procedures. For example, ultrasound may be used during liver surgery to visualize the vasculature of the liver. Ultrasound may be used for evaluating perfusion of the liver by estimating the volumetric flow through identified vascular features (e.g., vessels). Blood flow moves in three dimensions, but conventional ultrasound generally only detects flow in one dimension, such as whether the blood is flowing away from, or towards, the ultrasound probe and at what speed the blood is flowing. That is, what is actually measured with the ultrasound is the projection of the true flow onto the direction of the ultrasound beam. To derive the true flow it might be necessary to divide the measured flow with the cosine of the angle of the true flow and the direction of the beam. This process is called angle compensation. Generally, ultrasound systems may perform angle compensation of the flow in two dimensions by calculating the orientation of the vascular feature using the walls of the vascular feature provided the vessel locally runs strictly in the ultrasound plane. Having access to 3D or four dimensional (4D) imaging capabilities might allow for an exact angle compensation of the flow. However, ultrasound probes that include these capabilities are generally not available for laparoscopic ultrasound imaging due to their footprint not being accessible through laparoscopic ports.

### SUMMARY

This summary introduces concepts that are described in more detail in the detailed description. It should not be used to identify essential features of the claimed subject matter, nor to limit the scope of the claimed subject matter.

In an aspect, an ultrasound system may include transducer elements configured to generate ultrasound signals, transmit the ultrasound signals towards a region of interest of a subject, and receive echo signals reflected by the region of interest of the subject; an acoustic matching layer configured to match an impedance differential between the transducer elements and the subject; a backing layer configured to attenuate the ultrasound signals transmitted by the transducer elements; a memory configured to store instructions; and one or more processors configured to execute the instructions to: acquire ultrasound data of a vascular structure of a region of interest of a subject from an ultrasound probe; acquire tracking data of the ultrasound probe from a tracking system; generate a three-dimensional (3D) vascular model of the vascular structure using the ultrasound data and the tracking data; acquire angle-compensated flow information of the vascular structure using a flow imaging technique and the 3D vascular model of the vascular structure; and display the angle-compensated flow information of the vascular structure.

In another aspect, a method may include acquiring ultrasound data of a vascular structure of a region of interest of a subject from an ultrasound probe; acquiring tracking data of the ultrasound probe from a tracking system; generating a three-dimensional (3D) vascular model of the vascular structure using the ultrasound data and the tracking data; acquiring angle-compensated flow information of the vascular structure using a flow imaging technique and the 3D vascular model of the vascular structure; and displaying the angle-compensated flow information of the vascular structure.

In yet another aspect, a non-transitory computer-readable medium may store instructions that, when executed by one or more processors, cause the one or more processors to: acquire ultrasound data of a vascular structure of a region of interest of a subject from an ultrasound probe; acquire tracking data of the ultrasound probe from a tracking system; generate a three-dimensional (3D) vascular model of the vascular structure using the ultrasound data and the tracking data; acquire angle-compensated flow information of the vascular structure using a flow imaging technique and the 3D vascular model of the vascular structure; and display the angle-compensated flow information of the vascular structure.

### BRIEF DESCRIPTION OF DRAWINGS

FIG. 1 is a diagram of an example system for acquiring angle-compensated flow information using a 3D vascular model generated based on tracking data of an ultrasound probe.

FIG. 2 is a diagram of example components of an ultrasound system of FIG. 1.

FIG. 3 is a diagram of example components of an ultrasound probe of FIG. 2.

FIG. 4 is a diagram of example components of a tracking system of FIG. 1.

FIG. 5 is a flowchart of an example process for acquiring angle-compensated flow information using a 3D vascular model generated based on tracking data of an ultrasound probe.

FIG. 6 is a flowchart of an example process for acquiring angle-compensated flow information based on performing angle compensation on non-compensated flow information.

FIG. 7 is a flowchart of an example process for acquiring angle-compensated flow information based on controlling an ultrasound probe to steer an ultrasound beam.

FIG. 8 is flowchart of an example process 800 for determining a flow rate of blood flow through a vascular structure using a 3D vascular model.

FIG. 9 is a diagram of an example vascular structure of a region of interest of a subject and an ultrasound probe.

FIG. 10 is a diagram of an example segmentation map of a vascular structure generated using ultrasound data.

FIG. 11 is a diagram of an example 3D vascular model of a vascular structure of a region of interest of a subject.

FIG. 12 is a diagram of an example ultrasound image and a corresponding insonified portion of a 3D vascular model.

FIG. 13 is a diagram of an example process for training, deploying, and monitoring an artificial intelligence (AI) model.

### DETAILED DESCRIPTION

As addressed above, an ultrasound system may perform angle compensation using 2D images, each of which might not individually comprehensively or accurately account for the 3D orientation of a vascular structure and/or the 3D flow of blood. This is particularly important as a 3D ultrasound transducer or 4D ultrasound transducer might not be capable of being utilized for laparoscopy due to size constraints.

Some embodiments herein provide an ultrasound system that may acquire ultrasound data of a vascular structure of a region of interest of a subject from an ultrasound probe, and acquire tracking data of the ultrasound probe from a tracking system. The ultrasound system may generate a 3D vascular model of the vascular structure using the ultrasound data and the tracking data, and acquire angle-compensated flow information of the vascular structure using a flow imaging technique and the 3D vascular model of the vascular structure. The ultrasound system may display the angle-compensated flow information of the vascular structure.

By utilizing the 3D vascular model, the ultrasound system may acquire flow information that is angle-compensated based on the actual 3D orientation of the vascular structure. In this way, the angle-compensated flow information is more accurate than as compared to implementations that utilize 2D images to perform angle compensation. Further, by utilizing the tracking data, the ultrasound system may generate the vascular model using a relatively small form factor ultrasound probe. In this way, the embodiments here are applicable to laparoscopy as compared to implementations that would otherwise necessitate a 3D ultrasound system or 4D ultrasound system.

By providing more accurate volume flow estimates using intra-operative ultrasound, the embodiments herein may also assist perfusion quantification of, as an example, a transplanted liver. By being able to make an accurate perfusion estimate while the patient is still on the operating table, the embodiments herein can assist the surgeon in catching any abnormalities, and fixed them immediately, instead of having to re-hospitalize the patient after insertion of the transplanted liver.

In light of the foregoing, the embodiments herein provide an improvement in the technical field of ultrasound imaging and an improvement to ultrasound systems by providing for the generation of a 3D vascular model of a vascular structure and providing for angle compensation using the 3D vascular model. In this way, the angle-compensated flow information is more accurate than as compared to situations in which the 3D nature of the vascular structure and blood flow throughout the vascular structure is not accounted for. Further, the embodiments herein provide an improvement to surgical procedures by permitting more comprehensive and accurate determination of blood flow throughout the vasculature of a region of interest in real-time.

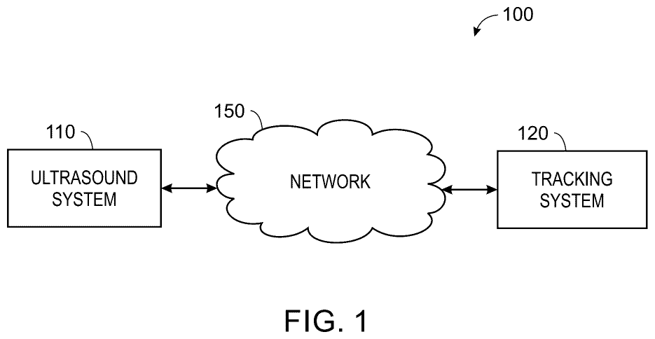

FIG. 1 is a diagram of an example system 100 for acquiring angle-compensated flow information using a 3D vascular model generated based on tracking data of an ultrasound probe. As shown in FIG. 1, the system 100 may include an ultrasound system 110, a tracking system 120, and a network 130.

The ultrasound system 110 may be configured to acquire ultrasound data of a vascular structure of a region of interest of a subject. For example, the ultrasound system 110 may be a 2D ultrasound system, a 3D ultrasound system, a 4D ultrasound system, a Doppler ultrasound system, or the like.

The tracking system 120 may be configured to acquire tracking data of an ultrasound probe of the ultrasound system 110. For example, the tracking system 120 may be an electromagnetic tracking system, an optical tracking system, an acoustic tracking system, an inertial tracking system, or the like.

The network 130 may permit communication between the ultrasound system 110 and the tracking system 120. For example, the network 130 may be a local area network (LAN), a wide area network (WAN), a metropolitan area network (MAN), a cellular network, a private network, an ad hoc network, an intranet, the Internet, a fiber optic-based network, or the like, and/or a combination of these or other types of networks.

The number and arrangement of the systems of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

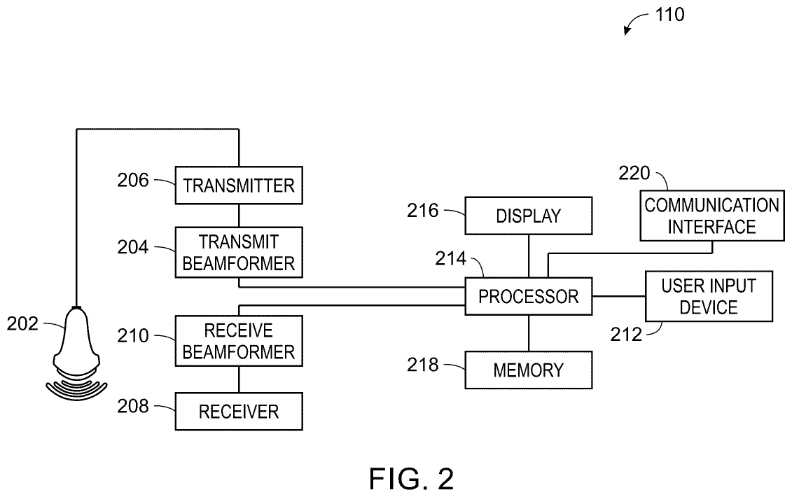

FIG. 2 is a diagram of example components of an ultrasound system 110 of FIG. 1. As shown in FIG. 2, the ultrasound system 110 may include an ultrasound probe 202, a transmit beamformer 204, a transmitter 206, a receiver 208, a receive beamformer 210, a user input device 212, a processor 214, a display 216, a memory 218, and a communication interface 220. The foregoing components may be connected via wired or wireless connections.

The ultrasound probe 202 may be configured to acquire ultrasound data. For example, the ultrasound probe 202 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 202 may be configured to generate ultrasound signals, emit the ultrasound signals towards a region of interest of a subject, receive echo ultrasound signals that are back-scattered from the region of interest of the subject, generate ultrasound data based on the echo ultrasound signals, and display the ultrasound data. The region of interest may be any region of the anatomy of a subject. The subject may be a person, an animal, a phantom, or the like.

The transmit beamformer 204 may be configured to apply delay times to electrical signals provided to the elements of the ultrasound probe 202 to focus corresponding ultrasound signals at the region of interest. The transmitter 206 may be configured to transmit electrical signals to the elements of the ultrasound probe 202 to drive the elements to emit ultrasound signals towards the region of interest. The elements may be configured to receive the electrical signals from the transmitter 206, convert the electrical signals into ultrasound signals, and emit the ultrasound signals towards the region of interest. The elements may be configured to receive echo ultrasound signals that are back-scattered by the region of interest, convert the echo ultrasound signals into electrical signals, and provide the electrical signals to the receiver 208. The receiver 208 may be configured to receive electrical signals from the elements, and provide the electrical signals to the receive beamformer 210. The receive beamformer 210 may apply delay times to the electrical signals received from the elements.

The user input device 212 may be configured to receive a user input, and provide the user input to the processor 214. For example, the user input device 212 may be a user interface, a touch screen display, a keyboard, a keypad, a mouse, a button, a switch, a microphone, or the like. Additionally, or alternatively, the user input device 212 may be configured to sense information. For example, the user input device 212 may sense information from an electro-magnetic positioning system, an inertial measurement system, an accelerometer, a gyroscope, an actuator, or the like.

The processor 214 may be configured to perform the operations as described herein. For example, a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 214 may be implemented in hardware, firmware, or a combination of hardware and software. The processor 214 may include one or more processors 214 configured to perform the operations described herein. For example, a single processor 214 may be configured to perform all of the operations described herein. Alternatively, multiple processors 214, collectively, may be configured to perform all of the operations described herein, and each of the multiple processors 214 may be configured to perform a subset of the operations described herein. For example, a first processor 214 may perform a first subset of the operations described herein, a second processor 214 may be configured to perform a second subset of the operations described herein, etc.

The processor 214 may be configured to control the ultrasound probe 202 to acquire ultrasound data. The processor 214 may be configured to control which of the elements are active, and control the shape of a beam emitted from the ultrasound probe 202. The processor 214 may generate ultrasound images for display. For example, the processor 214 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 216 may be configured to display information. For example, the display 216 may be a monitor, an LED display, a cathode ray tube, a projector display, a touchscreen, tablet computer, mobile phone, or the like. The display 216 may display ultrasound images based on the ultrasound data in real-time. For example, the display 216 may display the ultrasound images within one second, two seconds, five seconds, etc., of the ultrasound data being acquired by the ultrasound probe 202.

The memory 218 may be configured to store information and/or instructions for use by the processor 214. The memory 218 may be a non-transitory computer-readable medium. For example, a random access memory (RAM), a read only memory (ROM), and/or another type of dynamic or static storage device (e.g., a flash memory, a magnetic memory, and/or an optical memory) that stores information and/or instructions for use by the processor 214. The memory 218 may be configured to store instructions that, when executed by the processor 214, cause the processor 214 to perform the operations described herein.

The communication interface 220 may be configured to enable the processor 214 to communicate with other systems, such as via a wired connection, a wireless connection, or a combination of wired and wireless connections. For example, the communication interface 220 may include an Ethernet interface, an optical interface, a coaxial interface, an infrared interface, a radio frequency (RF) interface, a universal serial bus (USB) interface, a Wi-Fi interface, a cellular network interface, or the like.

The number and arrangement of the components of the ultrasound system 110 shown in FIG. 2 are provided as an example. In practice, the ultrasound system 110 may include additional components, fewer components, different components, or differently arranged components than those shown in FIG. 2. Additionally, or alternatively, a set of components (e.g., one or more components) of the ultrasound system 110 may perform one or more functions described as being performed by another set of components of the ultrasound system 110.

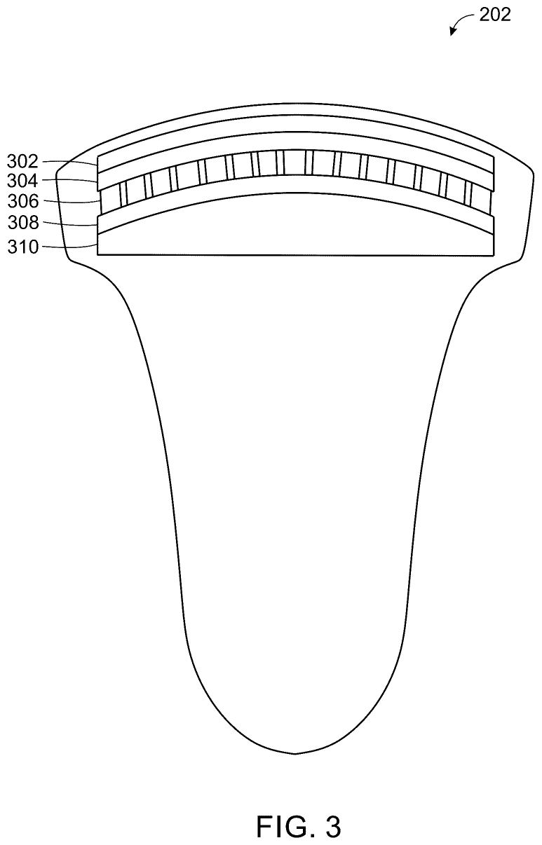

FIG. 3 is a diagram of example components of an ultrasound probe 202 of FIG. 2. As shown in FIG. 3, the ultrasound probe 202 may include a lens 302, an acoustic matching layer 304, transducer elements 306, an acoustic dematching layer 308, and a backing layer 310.

According to an embodiment, the lens 302 may be configured to direct an ultrasound signal towards the region of interest of the subject. For example, the lens 302 may be silicone, epoxy, rubber, or the like. According to an embodiment, the acoustic matching layer 304 may be configured to facilitate matching of an impedance differential that may exist between the relatively high impedance transducer elements 306 and the relatively low impedance subject. For example, the acoustic matching layer 304 may be graphite, plastic, resin, or the like. According to an embodiment, the transducer elements 306, respectively, may be configured to receive an element specific transmit signal, deform based on the element specific transmit signal, generate an ultrasound signal based on the deformation, and transmit the ultrasound signal towards a region of interest. Additionally, or alternatively, the transducer elements 306 may be configured to receive an echo signal reflected by or backscattered from the region of interest, deform based on the echo signal, generate an electrical signal based on the deformation, and transmit the electrical signal. For example, the transducer elements 306 may be piezoelectric materials, such as Pb(Mg_{1/3}Nb_{2/3})O₃—PbTiO₃ (“PMN-PT”), Pb(In_{1/2}Nb_{1/2})O₃−Pb(Mg_{1/3}Nb_{2/3})O₃—PbTiO₃ (“PIN-PMN-PT”), Pb(ZrTi) (“PZT”), or the like. According to an embodiment, the acoustic dematching layer 308 may be configured to decrease insertion losses and enhance a frequency bandwidth of the transducer elements 306. For example, the acoustic dematching layer 308 may be tungsten carbide, silicon carbide, or the like. According to an embodiment, the backing layer 310 may be configured to attenuate ultrasound signals directed from the transducer elements 306 in a direction opposite to the subject, and attenuate ultrasound signals deflected by a housing of the ultrasound probe 202. For example, the backing layer 310 may be an epoxy, a metal, or the like.

The number and arrangement of the components of the ultrasound probe 202 shown in FIG. 3 are provided as an example. In practice, the ultrasound probe 202 may include additional components, fewer components, different components, or differently arranged components than those shown in FIG. 3. Additionally, or alternatively, a set of components (e.g., one or more components) of the ultrasound probe 202 may perform one or more functions described as being performed by another set of components of the ultrasound probe 202.

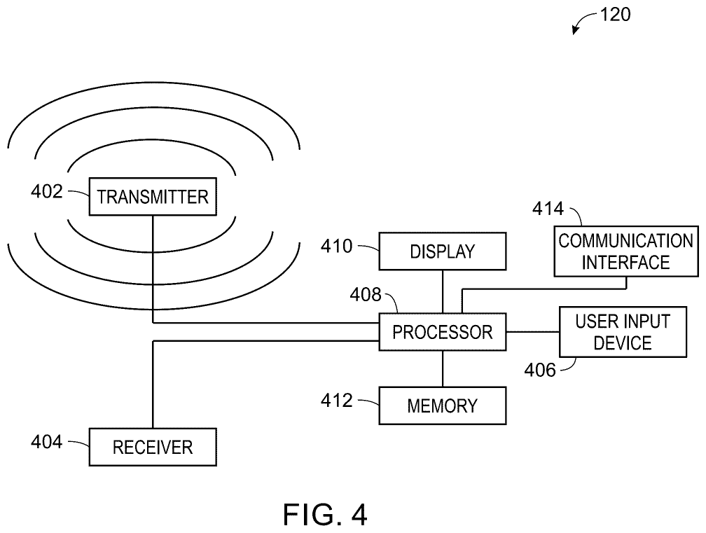

FIG. 4 is a diagram of example components of the tracking system 120 of FIG. 1. As shown in FIG. 4, the tracking system 120 may be an electromagnetic tracking system, and may include a transmitter 402, a receiver 404, a user input device 406, a processor 408, a display 410, a memory 412, and a communication interface 414.

The transmitter 402 may be configured to generate a magnetic field. The receiver 404 may be configured to output a signal in response to the magnetic field generated by the transmitter 402. The processor 408 may receive the output signal from the receiver 404, and acquire tracking data that identifies a position and/or an orientation of the receiver 404. The receiver 404 may be attached to, integrated with, provided in, etc., a tracked instrument. For example, according to an embodiment, the receiver 404 may be attached to the ultrasound probe 202 to track a position and/or an orientation of the ultrasound probe 202.

The user input device 406 may be configured to receive a user input, and provide the user input to the processor 408. For example, the user input device 406 may be a user interface, a touch screen display, a keyboard, a keypad, a mouse, a button, a switch, a microphone, or the like. Additionally, or alternatively, the user input device 406 may be configured to sense information. For example, the user input device 406 may sense information from an electro-magnetic positioning system, an inertial measurement system, an accelerometer, a gyroscope, an actuator, or the like.

The processor 408 may be configured to perform the operations as described herein. For example, the processor 154 may be a CPU, a GPU, an APU, a microprocessor, a microcontroller, a DSP, an FPGA, an ASIC, or the like. The processor 408 may be implemented in hardware, firmware, or a combination of hardware and software. The processor 408 may include one or more processors 408 configured to perform the operations described herein. For example, a single processor 408 may be configured to perform all of the operations described herein. Alternatively, multiple processors 408, collectively, may be configured to perform all of the operations described herein, and each of the multiple processors 408 may be configured to perform a subset of the operations described herein. For example, a first processor 408 may perform a first subset of the operations described herein, a second processor 408 may be configured to perform a second subset of the operations described herein, etc.

The processor 408 may be configured to control the transmitter 402 to acquire ultrasound data. The processor 408 may be configured to control excitations of the transmitter 402 to generate a magnetic field. The processor 408 may acquire tracking data based on controlling the transmitter 402.

The display 410 may be configured to display information. For example, the display 410 may be a monitor, an LED display, a cathode ray tube, a projector display, a touchscreen, tablet computer, mobile phone, or the like. The display 410 may display the tracking data in real-time. For example, the display 410 may display the tracking data within one second, two seconds, five seconds, etc., of the tracking data being acquired.

The memory 412 may be configured to store information and/or instructions for use by the processor 408. The memory 412 may be a non-transitory computer-readable medium. For example, the memory 412 may be a RAM, a ROM, a flash memory, a magnetic memory, an optical memory, or the like. The memory 412 may be configured to store instructions that, when executed by the processor 408, cause the processor 408 to perform the operations described herein.

The communication interface 414 may be configured to enable the processor 408 to communicate with other systems, such as via a wired connection, a wireless connection, or a combination of wired and wireless connections. For example, the communication interface 414 may include an Ethernet interface, an optical interface, a coaxial interface, an infrared interface, an RF interface, a USB interface, a Wi-Fi interface, a cellular network interface, or the like.

The number and arrangement of the components of the tracking system 120 shown in FIG. 4 are provided as an example. In practice, the tracking system 120 may include additional components, fewer components, different components, or differently arranged components than those shown in FIG. 4. Additionally, or alternatively, a set of components (e.g., one or more components) of the tracking system 120 may perform one or more functions described as being performed by another set of components of the tracking system 120.

Although FIG. 4 depicts the tracking system 120 as being an electromagnetic tracking system, it should be understood that the embodiments herein are applicable to other types of tracking systems, such as optical tracking systems, acoustic tracking systems, ultrasound tracking systems, or the like.

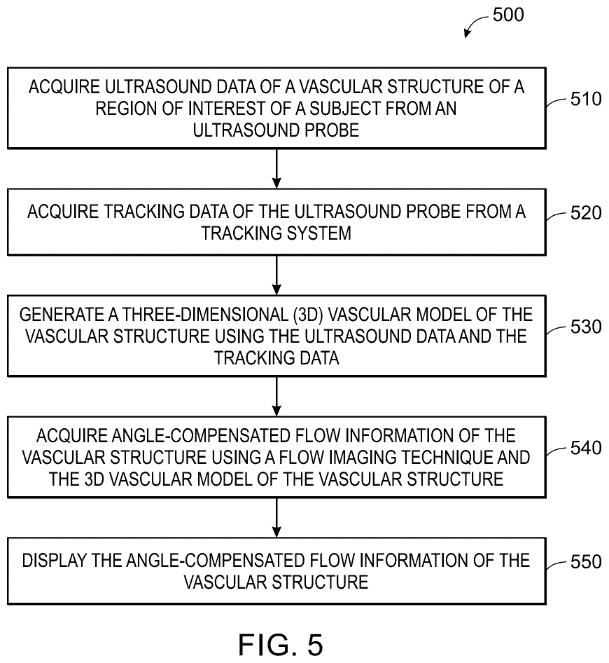

FIG. 5 is a flowchart of an example process 500 for acquiring angle-compensated flow information using a 3D vascular model generated based on tracking data of an ultrasound probe.

As shown in FIG. 5, the process 500 may include acquiring ultrasound data of a vascular structure of a region of interest of a subject from an ultrasound probe (operation 510). For example, the ultrasound system 110 may acquire ultrasound data of a vascular structure of a region of interest of a subject from the ultrasound probe 202. The ultrasound data may include 2D ultrasound data, 3D ultrasound data, 4D ultrasound data, or the like. The region of interest may be anatomical region of the subject. For example, the region of interest may be a liver, a heart, a lung, a pancreas, a brain, a neck, etc. The vascular structure may be any vascular structure in the region of interest. For example, the vascular structure may be a vessel (e.g., an artery, a vein, or a capillary), a valve, a tissue, an organ, or the like. The user may manipulate the ultrasound probe 202 with respect to the region of interest of the subject to cause the ultrasound probe 202 to acquire the ultrasound data of the vascular structure of the region of interest of the subject. The ultrasound data may be ultrasound data that is used to generate a 3D vascular model of the vascular structure. The ultrasound data may include a set of 2D images, a 3D image, a 4D image, or the like. The ultrasound data may include a threshold amount of data for generating the 3D vascular model. According to an embodiment, the user may perform a predetermined gesture with respect to the subject to acquire the ultrasound data. For example, the user may move the ultrasound probe 202 in a predetermined manner, in a predetermined set of directions, in a predetermined pattern, or the like, to permit the ultrasound system 110 to acquire a sufficient amount of the ultrasound data for generating the 3D vascular model. In some cases, the ultrasound system 110 may display scan guidance information that instructs the user to manipulate and/or orient the ultrasound probe 202 with respect to the subject to acquire the ultrasound data.

As further shown in FIG. 5, the process 500 may include acquiring tracking data of the ultrasound probe from a tracking system (operation 520). For example, the ultrasound system 110 may acquire tracking data of the ultrasound probe 202 from the tracking system 120. The tracking data may be data that identifies a position of the ultrasound probe 202 in a coordinate space. The coordinate space may be a coordinate space of the ultrasound system 110, a coordinate space of the tracking system 120, a coordinate space of an intraoperative location (e.g., a catheterization laboratory), or the like. The ultrasound system 110 may correlate the respective ultrasound data acquired for 3D vascular model generation with the respective tracking data. In this way, the ultrasound system 110 may use the vascular structure in the ultrasound data and the tracking data to generate the 3D vascular model, as described below.

As further shown in FIG. 5, the process 500 may include generating a three-dimensional (3D) vascular model of the vascular structure using the ultrasound data and the tracking data (operation 530). For example, the ultrasound system 110 may generate a 3D vascular model of the vascular structure using the ultrasound data and the tracking data. The 3D vascular model may be a 3D model of the vascular structure in the region of interest. Each point in the 3D vascular model may be correlated with a coordinate of the coordinate space. The 3D vascular model may depict the 3D orientation of the vascular structure in the region of interest.

According to an embodiment, the ultrasound system 110 may generate the 3D vascular model of the vascular structure based on identifying the vascular structure in the ultrasound data. For example, the ultrasound system 110 may identify the vascular structure in the ultrasound data using an image processing technique. For example, the image processing technique may be a segmentation technique, a pattern matching technique, a feature extraction technique, an image analysis technique, an edge detection technique, an image registration technique, or the like. In this case, the ultrasound system 110 may analyze the ultrasound data using the image processing technique, and identify the vascular structure based on the analysis. For example, the ultrasound system 110 may analyze the ultrasound data to identify the vascular structure.

As another example, the ultrasound system 110 may identify the vascular structure in the ultrasound data using an AI model. For example, the ultrasound system 110 may input the ultrasound data into the AI model, and identify the vascular structure based on an output of the AI model. The AI model may be a convolutional neural network (CNN) model, a residual neural network, a random forest model, a decision tree model, an artificial neural network (ANN), a Naïve Bayes model, a decision tree, a recurrent neural network (RNN), a logistic regression model, a support vector machine, or the like. In this case, the AI model may be trained to receive ultrasound data, analyze the ultrasound data to identify the vascular structure in the ultrasound data, and output information that identifies the vascular structure. The training data may include known ultrasound data that is correlated with known vascular structures.

As another example, the ultrasound system 110 may identify the vascular structure in the ultrasound data using preoperative imaging data of the region of interest acquired by a preoperative imaging system (e.g., a computed tomography (CT) system, a magnetic resonance imaging (MRI) system, or the like). For example, the ultrasound system 110 may register the ultrasound data with preoperative imaging data, of a preoperative imaging dataset, acquired by a preoperative imaging system that identifies the vascular structure, and identify the vascular structure based on registering the ultrasound data with the preoperative imaging data. In this case, the preoperative imaging data may identify the vascular structure. In an embodiment, the preoperative imaging data may be automatically analyzed to identify the vascular structure. Alternatively, the preoperative imaging data may be manually labelled with the vascular structure.

As another example, the ultrasound system 110 may identify the vascular structure in the ultrasound data using a user input received via the user input device 212. For example, a user of the ultrasound system 110 may interact with the user input device 212 to select the vascular structure that is to be modelled. The ultrasound system 110 may identify the vascular structure based on the selection.

As further shown in FIG. 5, the process 500 may include acquiring angle-compensated flow information of the vascular structure using a flow imaging technique and the 3D vascular model of the vascular structure (operation 540). For example, the ultrasound system 110 may acquire angle-compensated flow information of the vascular structure using a flow imaging technique and the 3D vascular model of the vascular structure.

The flow imaging technique may be power Doppler imaging, color velocity imaging, vector flow imaging, spectral Doppler imaging, or the like. Restated, the flow imaging technique may be any technique for which angle-compensated flow information is acquired by the ultrasound system 110. The angle-compensated flow information may be flow information that is compensated based on the actual orientation of the vascular structure, or that accounts for the actual orientation of the vascular structure.

According to an embodiment, the angle-compensated flow information may be information that identifies a direction of blood flow (or fluid flow) in the vascular structure. Additionally, or alternatively, the angle-compensated flow information may be information that identifies a velocity of blood flow in the vascular structure. Additionally, or alternatively, the angle-compensated flow information may be information that identifies blood velocity along a CW line that coincides with the vascular structure over time. Additionally, or alternatively, the angle-compensated flow information may be information that identifies blood velocity along a PW line and within a PW gate that coincide with the vascular structure over time. Additionally, or alternatively, the angle-compensated flow information may be information that identifies a flow rate of blood through the vascular structure.

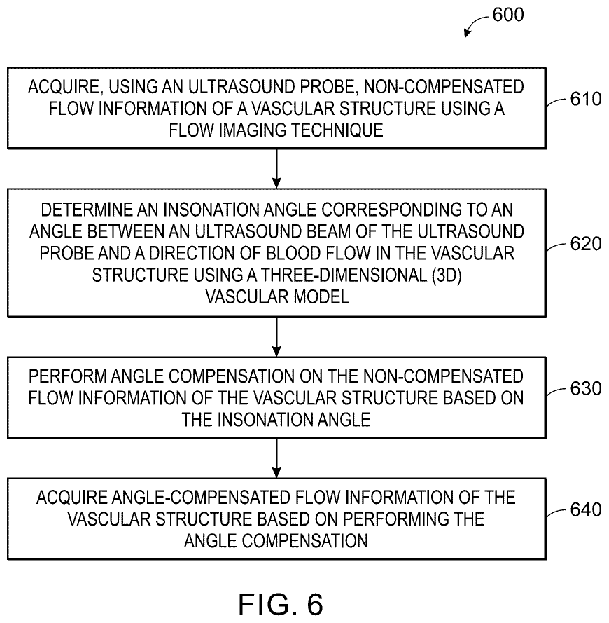

According to an embodiment, the ultrasound system 110 may acquire the angle-compensated flow information based on performing angle compensation on non-compensated flow information. For example, FIG. 6 is a process 600 for acquiring the angle-compensated flow information based on performing angle compensation on non-compensated flow information.

As shown in FIG. 6, the process 600 may include acquiring, using an ultrasound probe, non-compensated flow information of a vascular structure using a flow imaging technique (operation 610). For example, the ultrasound system 110 may acquire non-compensated flow information of a vascular structure using a flow imaging technique. The non-compensated flow information may be flow information that is not compensated based on the actual orientation of the vascular structure, or that does not account for the actual orientation of the vascular structure.

As further shown in FIG. 6, the process 600 may include determining an insonation angle corresponding to an angle between an ultrasound beam of the ultrasound probe 202 and a direction of blood flow in the vascular structure using a 3D vascular model (operation 620). For example, the ultrasound system 110 may determine a 3D orientation of the vascular structure using the 3D vascular model, and determine an insonation angle corresponding to an angle between an ultrasound beam of the ultrasound probe 202 and a direction of blood flow in the vascular structure based on the 3D orientation of the vascular structure.

As further shown in FIG. 6, the process 600 may include performing angle compensation on the non-compensated flow information of the vascular structure based on the insonation angle (operation 630). For example, the ultrasound system 110 may perform angle compensation on the non-compensated flow information of the vascular structure based on the insonation angle. As an example, the ultrasound system 110 may perform angle compensation by adjusting the non-compensated flow information based on the insonation angle to generate the angle-compensated flow information. In this way, the angle-compensated flow information more accurately reflects the actual flow of blood through the vascular structure.

As further shown in FIG. 6, the process 600 may include acquiring angle-compensated flow information of the vascular structure based on performing the angle compensation (operation 640). For example, the ultrasound system 110 may acquire angle-compensated flow information of the vascular structure based on performing the angle compensation. In this way, the angle-compensated flow information is more accurate than as compared to the non-compensated flow information because the angle-compensated flow information is compensated based on the insonation angle.

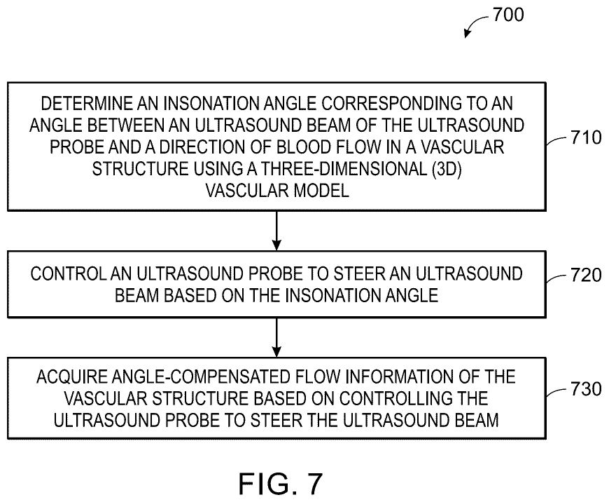

According to another embodiment, the ultrasound system 110 may acquire the angle-compensated flow information based on controlling the ultrasound probe 202 to steer an ultrasound beam. For example, FIG. 7 is a process 700 for acquiring the angle-compensated flow information based on controlling the ultrasound probe 202 to steer an ultrasound beam.

As shown in FIG. 7, the process 700 may include determining an insonation angle corresponding to an angle between an ultrasound beam of the ultrasound probe 202 and a direction of blood flow in a vascular structure using a 3D vascular model (operation 710). For example, the ultrasound system 110 may determine a 3D orientation of the vascular structure using the 3D vascular model, and determine an insonation angle corresponding to an angle between an ultrasound beam of the ultrasound probe 202 and a direction of blood flow in the vascular structure based on the 3D orientation of the vascular structure.

As further shown in FIG. 7, the process 700 may include controlling the ultrasound probe 202 to steer an ultrasound beam based on the insonation angle (operation 720). For example, the ultrasound system 110 may control the ultrasound probe 202 to steer the ultrasound beam based on the insonation angle. According to an embodiment, the ultrasound system 110 may control the ultrasound probe 202 to steer the ultrasound beam to reduce, or minimize, the insonation angle.

As further shown in FIG. 7, the process 700 may include acquiring angle-compensated flow information of the vascular structure based on controlling the ultrasound probe 202 to steer the ultrasound beam (operation 730). For example, the ultrasound system 110 may acquire angle-compensated flow information of the vascular structure based on controlling the ultrasound probe 202 to steer the ultrasound beam. In this way, the angle-compensated flow information more accurately reflects the velocity of blood flow because the angle-compensated flow information is acquired based on the insonation angle in a manner that reduces, or minimizes, the insonation angle.

According to an embodiment, the ultrasound system 110 may acquire angle-compensated flow information of the vascular structure at a particular position of the vascular structure. For example, the ultrasound system 110 may acquire angle-compensated flow information of the vascular structure at a particular position that coincides with the ultrasound beam of the ultrasound probe 202. As another example, the ultrasound system 110 may acquire angle-compensated flow information of the vascular structure at a particular set of positons that correspond to a bounding box displayed on the display 216 of the ultrasound system 110. For instance, the display 116 may display a bounding box that identifies a set of positions of a displayed ultrasound image for which flow information is to be acquired. Further, the ultrasound system 110 may determine a set of positions of the vascular structure that coincide with the bounding box, and acquire the angle-compensated flow information of the vascular structure at the particular set of positions. As another example, the ultrasound system 110 may acquire angle-compensated flow information of the vascular structure at a particular set of positons that correspond to a CW line. For example, the display 216 may display a CW line that identifies a set of positions of a displayed ultrasound image for which flow information is to be acquired. Further, the ultrasound system 110 may determine a set of positions of the vascular structure that coincide with the CW line, and acquire the angle-compensated flow information of the vascular structure at the particular set of positions. As another example, the ultrasound system 110 may acquire angle-compensated flow information of the vascular structure at a particular set of positons that correspond to a PW line and a PW gate. For example, the display 216 may display a PW line and a PW gate that identifies a set of positions of a displayed ultrasound image for which flow information is to be acquired. Further, the ultrasound system 110 may determine a set of positions of the vascular structure that coincide with the PW line and the PW gate, and acquire the angle-compensated flow information of the vascular structure at the particular set of positions.

As further shown in FIG. 5, the process 500 may include displaying the angle-compensated flow information of the vascular structure (operation 550). For example, the ultrasound system 110 may display the angle-compensated flow information. As an example, the ultrasound system 110 may display an ultrasound image and may overlay the angle-compensated flow information on the ultrasound image. According to an embodiment, the ultrasound system 110 may adjust a color, a shading, a brightness, a hue, or the like, of the vascular structure based on the angle-compensated flow information. Additionally, or alternatively, the ultrasound system 110 may display the angle-compensated flow information in the form of power spectra that identifies fluid velocity along a CW line that coincides with the vascular structure over time. Additionally, or alternatively, the ultrasound system 110 may display the angle-compensated flow information in the form of power spectra that identifies fluid velocity along a PW line and within a PW gate that coincide with the vascular structure over time. According to an embodiment, the angle-compensated flow information may be information that identifies a direction of blood flow, a velocity of blood flow, a flow rate of blood flow, or the like. The values may be median values, average values, maximum values, minimum values, or the like.

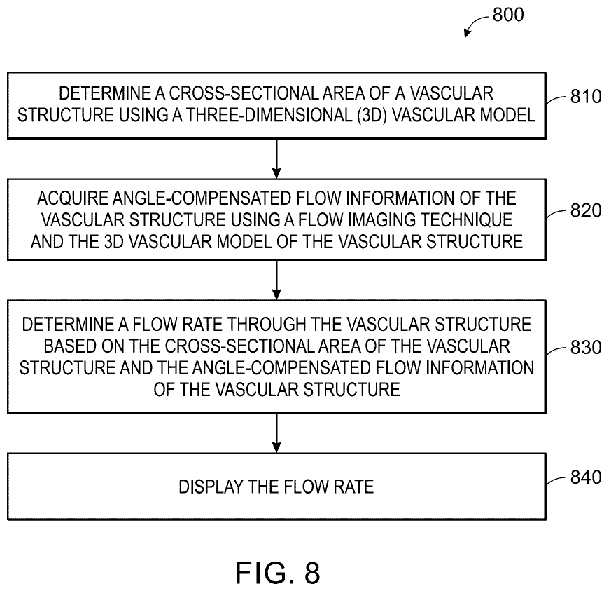

FIG. 8 is flowchart of an example process 800 for determining a flow rate of blood flow through a vascular structure using a 3D vascular model. As shown in FIG. 8, the process 800 may include determining a cross-sectional area of a vascular structure using a 3D vascular model (operation 810). For example, the ultrasound system 110 may determine a cross-sectional area of a vascular structure using a 3D vascular model. The ultrasound system 110 may determine one or more positions for which flow rate through the vascular structure is to be measured. For example, the one or more positons may correspond to one or more positions of the vascular structure that coincide with an ultrasound beam of the ultrasound probe 202. As another example, the one or more positons may correspond to one or more positions of the vascular structure that are included in a bounding box displayed on the display 216 of the ultrasound system 110. For instance, the display 116 may display a bounding box that identifies a set of positions of a displayed ultrasound image for which flow rate is to be acquired. As another example, the one or more positons may correspond to one or more positions of the vascular structure that coincide with a CW line, or that coincide with a PW line and are included within a PW gate. As another example, the one or more positons may correspond to one or more positions of the vascular structure that are selected by a user via a user interface. As another example, the one or more positons may correspond to one or more positions of the vascular structure that are shown on an ultrasound image displayed via the display 216. The ultrasound system 110 may determine a respective 3D orientation of the vascular structure using the 3D vascular model at the one or more positions, and determine one or more planes that are perpendicular to the vascular structure based on the 3D orientation of the vascular structure. The ultrasound system 110 may determine respective cross-sectional areas of the one or more planes using the 3D vascular model, and determine the cross-sectional area based on the respective cross-sectional areas. For example, the ultrasound system 110 may determine an average cross-sectional area, a median cross-sectional area, a minimum cross-sectional area, a maximum cross-sectional area, or the like. As further shown in FIG. 8, the process 800 may include acquiring angle-compensated flow information of the vascular structure using a flow imaging technique and the 3D vascular model of the vascular structure (operation 820). For example, the ultrasound system 110 may acquire angle-compensated flow information of the vascular structure using a flow imaging technique and the 3D vascular model of the vascular structure. The ultrasound system 110 may acquire the angle-compensated flow information of the vascular structure at the one or more positions in a similar manner as described above in connection with operation 540 of FIG. 5. As further shown in FIG. 8, the process 800 may include determining a flow rate through the vascular structure based on the cross-sectional area of the vascular structure and the angle-compensated flow information of the vascular structure (operation 830). For example, the ultrasound system 110 may determine a flow rate through the vascular structure based on the cross-sectional area of the vascular structure and the angle-compensated flow information of the vascular structure. For instance, the ultrasound system 110 may determine a flow rate based on a velocity of blood flow and the cross-sectional area of the vascular structure. As further shown in FIG. 8, the process 800 may include displaying the flow rate (operation 840). For example, the ultrasound system 110 may display the flow rate via the display 216.

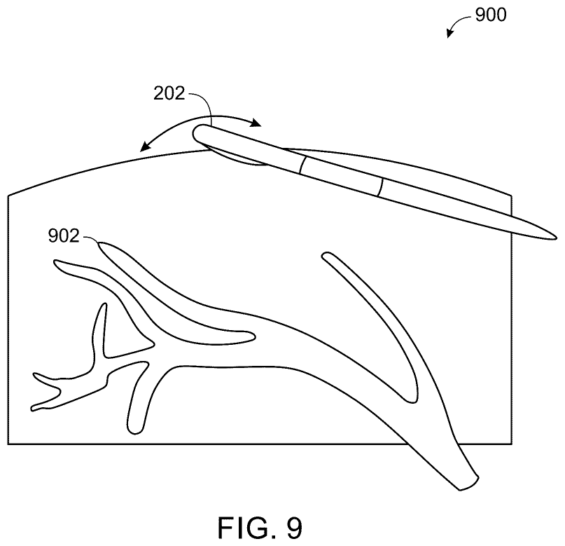

FIG. 9 is a diagram 900 of an example vascular structure of a region of interest of a subject and an ultrasound probe. For example, as shown in FIG. 9, a region of interest may include a vascular structure 902. The ultrasound probe 202 may be manipulated with respect to the subject to acquire ultrasound data of the vascular structure of the region of interest of the subject but especially for laparoscopic surgery the range of movement may be limited, so that it is difficult or impossible to have the desired vessel running completely within the imaging plane.

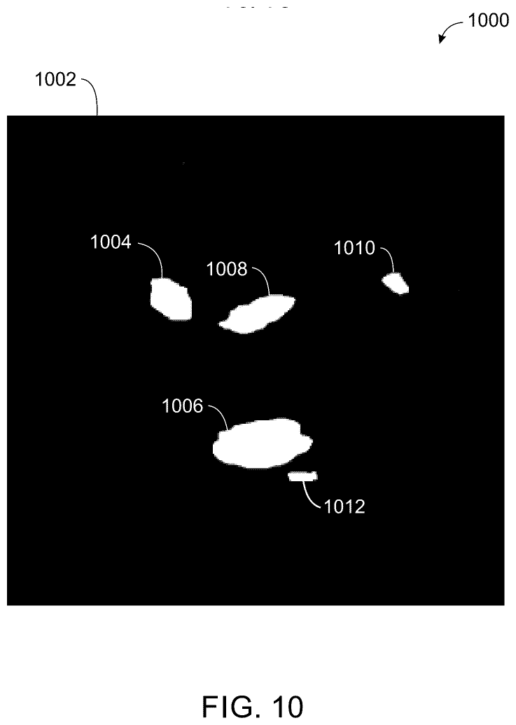

FIG. 10 is a diagram 1000 of an example segmentation map of a vascular structure generated using ultrasound data. As shown in FIG. 10, the ultrasound system 110 may generate a segmentation map 1002 that includes data that identifies segmented portions of the vascular structure. For example, as shown, the segmentation map 1002 may include a first segmented portion 1004, a second segmented portion 1006, a third segmented portion 1008, a fourth segmented portion 1010, and a fifth segmented portion 1012 that correspond to a segmented vascular structure.

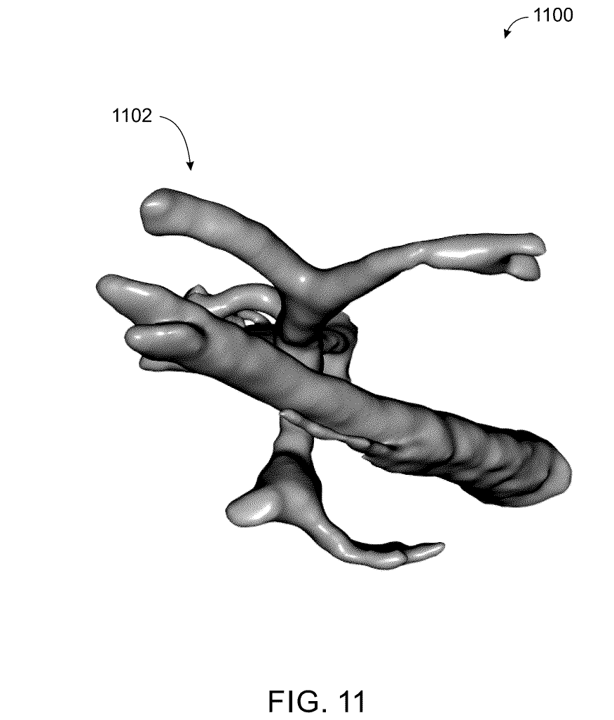

FIG. 11 is a diagram 1100 of an example 3D vascular model of a vascular structure of a region of interest of a subject. As shown in FIG. 11, the ultrasound system 110 may generate a 3D vascular model 1102 of the vascular structure that identifies the 3D orientation of the vascular structure.

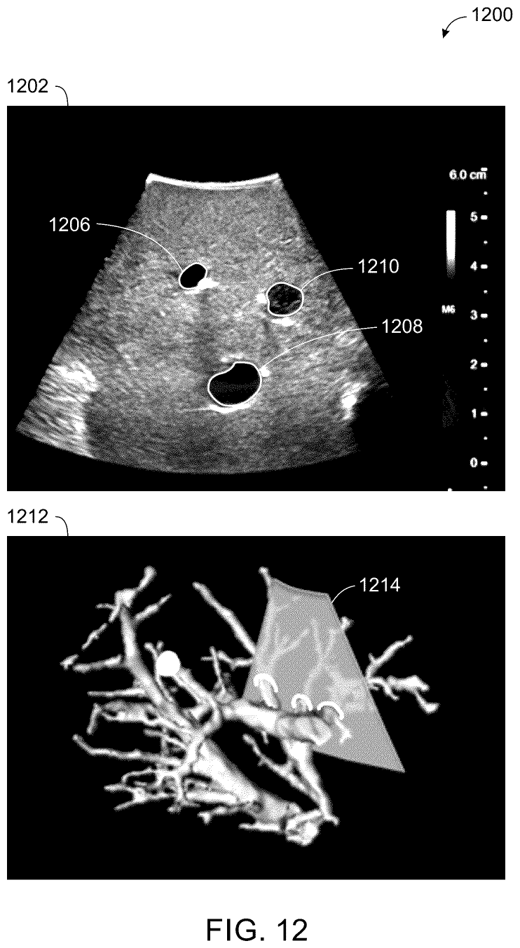

FIG. 12 is a diagram 1200 of an example ultrasound image and a corresponding insonified portion of a 3D vascular model. As shown in FIG. 12, the ultrasound system 110 may display an ultrasound image 1202 of the region of interest of the subject. As shown, the ultrasound image 1202 may depict a first portion 1206 of a vascular structure, a second portion 1208 of the vascular structure, and a third portion 1210 of the vascular structure. It should be understood that the portions may be associated with a same underlying vascular structure, or may be distinct vascular structures. As further shown in FIG. 11, the ultrasound image 1202 may correspond to a plane 1214 of an ultrasound beam of the ultrasound probe 202. The plane 1214 may corresponds to an insonified portion of a 3D vascular model 1212.

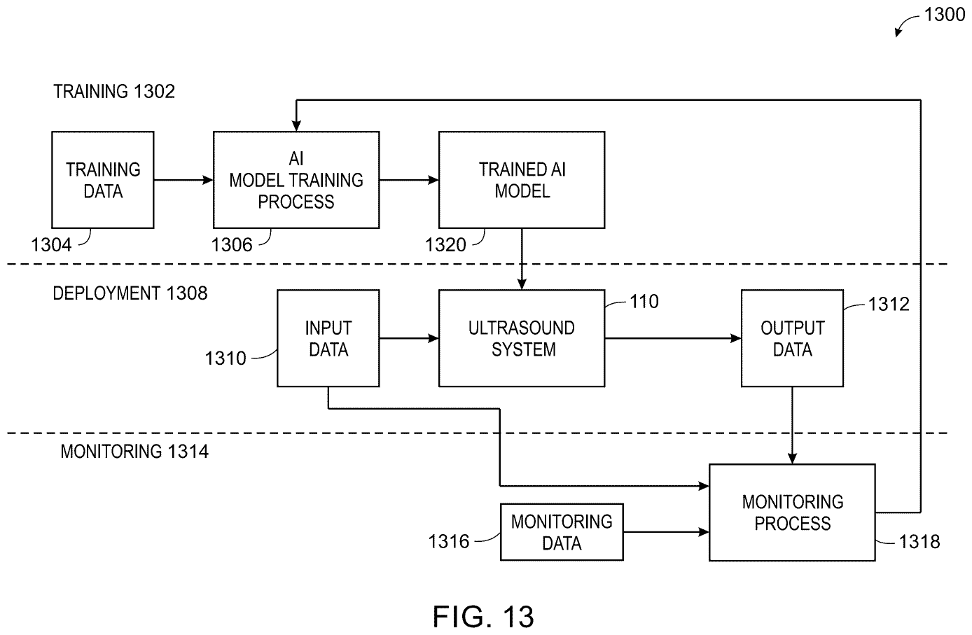

FIG. 13 is a diagram of an example process 1300 for training, deploying, and monitoring an AI model. According to an embodiment, the ultrasound system 110 may use one or more AI models 1320. The one or more AI models 1320 may be associated with a training phase 1302, a deployment phase 1308, and a monitoring phase 1314. In the training phase 1302, the ultrasound system 110 (or an external system) may receive and process training data 1304 to generate a trained model 1320. The training data 1304 may be generated, received, or otherwise obtained from internal and/or external resources.

Generally, the trained AI model 1320 may include a set of variables (e.g., nodes, neurons, filters, or the like) that are tuned (e.g., weighted, biased, or the like) to different values via the application of the training data 1304. According to an embodiment, the training process may employ supervised, unsupervised, semi-supervised, and/or reinforcement learning processes to train the model 1320. According to an embodiment, a portion of the training data 1304 may be withheld during training and/or used to validate the trained model 1320.

For supervised learning processes, the training data 1304 may include labels or scores that may facilitate the training process by providing a ground truth. For example, the labels or scores may indicate an output of the AI model 1320. Training may proceed by feeding the training data 1304 into the AI model 1320. The AI model 1320 may have variables set at initialized values (e.g., at random, based on Gaussian noise, based on pre-trained values, or the like). The AI model 1020 may generate an output based on the training data 1304 being input to the AI model 1320. The output may be compared with the corresponding label or score (e.g., the ground truth) indicating the known output, which may then be back-propagated through the AI model 1320 to adjust the values of the variables. This process may be repeated for a plurality of samples at least until a determined loss or error is below a predefined threshold. According to an embodiment, some of the training data 1304 may be withheld and used to further validate or test the trained AI model 1320.

For unsupervised learning processes, the training data 1304 may not include pre-assigned labels or scores to aid the training process. Instead, unsupervised learning processes may include clustering, classification, or the like, to identify naturally occurring patterns in the training data 1304. As an example, the training data 1304 may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data 1304 with pre-assigned labels or scores and training data without pre-assigned labels or scores may be used to train the AI model 1320.

When reinforcement learning is employed, an agent (e.g., an algorithm) may be trained to make a decision from the training data 1304 through trial and error. For example, based on making a decision, the agent may then receive feedback (e.g., a positive reward if the prediction was above a predetermined threshold), adjust its next decision to maximize the reward, and repeat until a loss function is optimized.

After being trained, the trained model 1320 may be stored and subsequently applied by the ultrasound system 110 during the deployment phase 1308. For example, during the deployment phase 1308, the trained AI model 1320 executed by the ultrasound system 110 may receive input data. During the deployment phase 1308, the trained AI model 1320 may perform one or more operations as described in connection with FIGS. 5, 6, 7, and/or 8.

After being deployed, the trained AI model 1320 may be monitored during the monitoring phase 1314. For example, during the monitoring phase 1314, the AI model 1320 may generate monitoring data that is used to monitor the trained AI model 1320. The monitoring data may include data that identifies an output as determined by an operator. During the monitoring phase 1314, monitoring data may be analyzed along with the predicted output data and input data to determine an accuracy of the trained AI model 1320. According to an embodiment, based on the analysis, the process may return to the training phase 1302, where values of one or more variables of the AI model 1320 may be adjusted to improve the accuracy of the AI model 1320.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. An ultrasound system comprising:
transducer elements configured to generate ultrasound signals, transmit the ultrasound signals towards a region of interest of a subject, and receive echo signals reflected by the region of interest of the subject;
an acoustic matching layer configured to match an impedance differential between the transducer elements and the subject;
a backing layer configured to attenuate the ultrasound signals transmitted by the transducer elements;
a memory configured to store instructions; and
one or more processors configured to execute the instructions to:
acquire ultrasound data of a vascular structure of the region of interest of the subject from an ultrasound probe;
acquire tracking data of the ultrasound probe from a tracking system;
generate a three-dimensional (3D) vascular model of the vascular structure using the ultrasound data and the tracking data;
acquire angle-compensated flow information of the vascular structure using a flow imaging technique and the 3D vascular model of the vascular structure; and
display the angle-compensated flow information of the vascular structure.

2. The ultrasound system of claim 1, wherein the one or more processors are further configured to:
acquire non-compensated flow information of the vascular structure of the region of interest of the subject from the ultrasound probe;
determine an insonation angle corresponding to an angle between an ultrasound beam of the ultrasound probe and a direction of blood flow in the vascular structure using the 3D vascular model; and
perform angle compensation on the non-compensated flow information of the vascular structure based on the insonation angle to acquire the angle-compensated flow information of the vascular structure.

3. The ultrasound system of claim 1, wherein the one or more processors are further configured to:
determine an insonation angle corresponding to an angle between an ultrasound beam of the ultrasound probe and a direction of blood flow in the vascular structure using the 3D vascular model; and
control the ultrasound probe to steer the ultrasound beam based on the insonation angle to acquire the angle-compensated flow information of the vascular structure.

4. The ultrasound system of claim 1, wherein the one or more processors are further configured to:
determine one or more planes that are perpendicular to a longitudinal axis of the vascular structure using the 3D vascular model;
determine a cross-sectional area of the vascular structure based on the one or more planes; and
determine a flow rate through the vascular structure based on the angle-compensated flow information and the cross-sectional area.

5. The ultrasound system of claim 1, wherein the one or more processors are further configured to generate the 3D vascular model using a segmentation technique.

6. The ultrasound system of claim 1, wherein the one or more processors are further configured to generate the 3D vascular model using an artificial intelligence (AI) model.

7. The ultrasound system of claim 1, wherein the one or more processors are further configured to:
determine an insonation angle based on an orientation of the vascular structure in the 3D vascular model of the vascular structure; and
acquire the angle-compensated flow information of the vascular structure using the flow imaging technique based on the insonation angle.

8. A method comprising:
acquiring ultrasound data of a vascular structure of a region of interest of a subject from an ultrasound probe;
acquiring tracking data of the ultrasound probe from a tracking system;
generating a three-dimensional (3D) vascular model of the vascular structure using the ultrasound data and the tracking data;
acquiring angle-compensated flow information of the vascular structure using a flow imaging technique and the 3D vascular model of the vascular structure; and
displaying the angle-compensated flow information of the vascular structure.

9. The method of claim 8, further comprising:
acquiring non-compensated flow information of the vascular structure of the region of interest of the subject from the ultrasound probe;
determining an insonation angle corresponding to an angle between an ultrasound beam of the ultrasound probe and a direction of blood flow in the vascular structure using the 3D vascular model; and
performing angle compensation on the non-compensated flow information of the vascular structure based on the insonation angle to acquire the angle-compensated flow information of the vascular structure.

10. The method of claim 8, further comprising:
determining an insonation angle corresponding to an angle between an ultrasound beam of the ultrasound probe and a direction of blood flow in the vascular structure using the 3D vascular model; and
controlling the ultrasound probe to steer the ultrasound beam based on the insonation angle to acquire the angle-compensated flow information of the vascular structure.

11. The method of claim 8, further comprising:
determining one or more planes that are perpendicular to a longitudinal axis of the vascular structure in the 3D vascular model;
determining a cross-sectional area of the vascular structure based on the one or more planes; and
determining a flow rate through the vascular structure based on the angle-compensated flow information and the cross-sectional area.

12. The method of claim 8, wherein the generating the 3D vascular model comprises generating the 3D vascular model using a segmentation technique.

13. The method of claim 8, wherein the generating the 3D vascular model comprises generating the 3D vascular model using an artificial intelligence (AI) model.

14. The method of claim 8, further comprising:
determining an insonation angle based on an orientation of the vascular structure in the 3D vascular model of the vascular structure; and
acquiring the angle-compensated flow information of the vascular structure using the flow imaging technique based on the insonation angle.

15. A non-transitory computer-readable medium storing instructions that, when executed by one or more processors, cause the one or more processors to:
acquire ultrasound data of a vascular structure of a region of interest of a subject from an ultrasound probe;
acquire tracking data of the ultrasound probe from a tracking system;
generate a three-dimensional (3D) vascular model of the vascular structure using the ultrasound data and the tracking data;
acquire angle-compensated flow information of the vascular structure using a flow imaging technique and the 3D vascular model of the vascular structure; and
display the angle-compensated flow information of the vascular structure.

16. The non-transitory computer-readable medium of claim 15, wherein the instructions further cause the one or more processors to:
acquire non-compensated flow information of the vascular structure of the region of interest of the subject from the ultrasound probe;
determine an insonation angle corresponding to an angle between an ultrasound beam of the ultrasound probe and a direction of blood flow in the vascular structure using the 3D vascular model; and
perform angle compensation on the non-compensated flow information of the vascular structure based on the insonation angle to acquire the angle-compensated flow information of the vascular structure.

17. The non-transitory computer-readable medium of claim 15, wherein the instructions further cause the one or more processors to:
determine an insonation angle corresponding to an angle between an ultrasound beam of the ultrasound probe and a direction of blood flow in the vascular structure using the 3D vascular model; and
control the ultrasound probe to steer the ultrasound beam based on the insonation angle to acquire the angle-compensated flow information of the vascular structure.

18. The non-transitory computer-readable medium of claim 15, wherein the instructions further cause the one or more processors to:
determine one or more planes that are perpendicular to a longitudinal axis of the vascular structure in the 3D vascular model;
determine a cross-sectional area of the vascular structure based on the one or more planes; and
determine a flow rate through the vascular structure based on the angle-compensated flow information and the cross-sectional area.

19. The non-transitory computer-readable medium of claim 15, wherein the instructions further cause the one or more processors to:
determine an insonation angle based on an orientation of the vascular structure in the 3D vascular model of the vascular structure; and
acquire the angle-compensated flow information of the vascular structure using the flow imaging technique based on the insonation angle.

20. The non-transitory computer-readable medium of claim 15, wherein the instructions further cause the one or more processors to generate the 3D vascular model using a segmentation technique.
